(19) United States
(12) Patent Application Publication
(10) Pub. No.: US 2017/0042448 A1
(43) Pub. Date: Feb. 16, 2017

(54) METHOD AND APPARATUS FOR ESTIMATING THE FLUID CONTENT OF PART OF THE BODY OF A SUBJECT

(71) Applicant: KONINKLIJKE PHILIPS N.V., EINDHOVEN (NL)

(72) Inventors: Silviu Dovancescu, Aachen (DE); Ronaldus Maria Aarts, Geldrop (NL); Rick Bezemer, Amsterdam Zuidoost (NL)

(21) Appl. No.: 15/305,216

(22) PCT Filed: May 7, 2015
(86) PCT No.: PCT/EP2015/060087

§ 371 Date: Oct. 19, 2016

(30) Foreign Application Priority Data
May 7, 2014 (EP) 14167414.3

(51) Int. Cl. A61B 5/053; A61B 5/00

(52) U.S. Cl. CPC A61B 5/0537; A61B 5/4878; A61B 5/6824; A61B 5/6825; A61B 5/6828; A61B 5/6829

(57) ABSTRACT

There is provided an apparatus for estimating the fluid content in a subject, the apparatus comprising a control unit that is configured to obtain a measurement of the bioimpedance of a first limb of the subject; obtain a measurement of the bioimpedance of a second limb of the subject; obtain a measurement of the bioimpedance of a segment of the body that includes at least the first limb and the second limb; and determine a measure of the fluid content in the first limb using the bioimpedance measurement of the first limb, the bioimpedance measurement of the second limb and the bioimpedance measurement of the segment of the body that includes at least the first limb and the second limb.

### TECHNICAL FIELD OF THE INVENTION

The invention relates to a method and apparatus for estimating the fluid content of part of the body of a subject, and in particular relates to a method and apparatus for estimating the fluid content (e.g. extracellular fluid, intracellular fluid or both) of one or more limbs of the subject from bioimpedance measurements.

### BACKGROUND TO THE INVENTION

Peripheral edema in the lower forearms and hands and/or lower legs and feet is a common complication in several patient populations, including patients with heart failure, nephrotic syndrome, liver cirrhosis, diabetes, hypertension and patients who had lymph surgery (e.g. as part of breast cancer surgery). Furthermore, pregnancies are often complicated by hypertension which also results in peripheral edema. A device that measures peripheral edema formation would provide an early warning to these patients to the potential requirement of an intervention.

Measurements of the bioimpedance of part of a subject's body provides a low-cost and non-invasive technique for detecting fluid content in the body. The principle underlying this technique is the fact that the electrical impedance (resistance and reactance) of biological tissue is directly linked to the hydration and water content of the tissue, namely intra-cellular and extra-cellular water. Therefore, measurements of the electrical properties of the tissue can indicate the amount of fluid present in that part of the body. Bioimpedance measurements can be used to determine the total amount of water in the body and the body composition (i.e. fat and fat free mass).

WO 00/079255 describes a method and device for measuring tissue edema in which measurements of bioelectrical impedance of first and second anatomical regions in a subject are made using a single low frequency alternating current and the measurements are analyzed to obtain an indication of the presence of tissue edema. The analysis may include the step of dividing the lesser result of the two measurements into the greater result of the two measurements to obtain a product or quotient. In this document, the first anatomical region and second anatomical region may be of the same type (e.g., the left and right legs) provided that at least one of the anatomical regions is unaffected by tissue edema. Alternatively, the first and second anatomical regions may be dissimilar (e.g., one arm and one leg) provided that at least one of the anatomical regions is unaffected by tissue edema.

WO 2005/122888 describes a method of detecting tissue edema in a subject in which a measured impedance is determined for first and second body segments; an index indicative of a ratio of the extra-cellular fluid to intra-cellular fluid for each of the segments is determined from measurements of impedance over four or more frequencies, and an index ratio is determined from the index for each of the first and second body segments and the presence, absence or degree of tissue edema is determined based on the index ratio. The first and second body segments are typically different types of body segment (e.g. an arm and a leg).

One of the main problems with bioimpedance measurements for body, limb, or tissue water/fluid content (edema) assessment is the lack of impedance values for ‘normal’ water content, and reproducibility of measurements. The patent applications referenced above address the first of these problems by looking to account for the inter-measurement variability (e.g. by finding a ratio of intracellular water to extracellular water per measurement) and the inter-patient variability (e.g. by finding a ratio of affected tissue to non-affected tissue/limb).

The reproducibility of the measurements is significantly affected by the need for consistent placement of the electrodes for each measurement. Typically, electrodes are placed 20-30 cm apart on each limb (e.g. on the lower legs or forearms). If electrodes on the different limbs are placed at slightly different locations, a measurement error of 10-20% can easily arise, which obviously reduces the accuracy and reproducibility. This inaccuracy increases when sequential measurements are performed on different days to track the potential formation of edema.

Therefore there is a need for an improved method and apparatus for estimating the fluid content of part of the body of a subject that has good versatility, minimal complexity and improved reproducibility and reliability compared to the conventional techniques.

### SUMMARY OF THE INVENTION

The invention provides that, rather than obtain bioimpedance measurements of separate (non-overlapping') body segments as in WO 00/079255 and WO 2005/122888, bioimpedance measurements are made for ‘overlapping’ body segments, and the measurements are used to assess the fluid content in the body segments. The segments are overlapping in the sense that one of the segments includes another one of the segments. For example, separate bioimpedance measurements can be made for one or both legs and for the whole lower body of the subject (e.g. by measuring the bioimpedance from the left foot to the right foot). The lower body segment thus overlaps the left leg and right leg segments. Other overlapping segments can comprise one or both arms of the subject and the upper body of the subject, or one or both of the left/right limbs and the left/right side of the body (which includes both of the left/right limbs).

By measuring the bioimpedance of overlapping body segments, it is possible to use only two electrodes for current injection rather than four as in WO 00/079255 and WO 2005/122888, which reduces the number of electrodes that can be placed in the wrong position when repeating a measurement (and thus increases the reliability by a factor two). In addition, when overlapping body segments are used it is still possible to express the fluid content of left and right limbs with respect to each other and with respect to the fluid content of the total upper or lower body. This makes the comparison between fluid in the left and right limbs more accurate.

Making bioimpedance measurements of overlapping body segments makes the measurements more versatile as it allows objective tracking of edema formation when, for example, it occurs in both legs (e.g. in the case of heart failure, nephrotic syndrome, liver cirrhosis, diabetes, hypertension, and pregnancy) rather than in only one body segment (e.g. in the case of lymphedema). By contrast, WO 00/079255 and WO 2005/122888 require a reference measurement on a similar body segment (e.g. left leg versus right leg) provided the similar body segment is unaffected by the edema, or on a dissimilar body segment (e.g. left leg versus left arm) if the similar segment is affected by the edema. In the latter case, as the bioimpedance measurements are made on dissimilar body segments, the interpretation of the measurements and obtained values for extracellular fluid is cumbersome.

A further advantage of making bioimpedance measurements of overlapping segments is that the measurements can be made at the same time.

According to a first specific aspect of the invention, there is provided an apparatus for estimating the fluid content in a subject, the apparatus comprising a control unit that is configured to obtain a measurement of the bioimpedance of a first limb of the subject; obtain a measurement of the bioimpedance of a second limb of the subject; obtain a measurement of the bioimpedance of a segment of the body that includes at least the first limb and the second limb; and determine a measure of the fluid content in the first limb using the bioimpedance measurement of the first limb, the bioimpedance measurement of the second limb and the bioimpedance measurement of the segment of the body that includes at least the first limb and the second limb.

In some embodiments the control unit is configured to determine a measure of the fluid content in the first limb by normalising the bioimpedance measurements for the first and second limbs using the bioimpedance measurement for the body segment that includes at least the first limb and the second limb.

In other embodiments the control unit is configured to determine a measure of the fluid content in the first limb by determining the amount of extracellular fluid and/or intracellular fluid in the first and second limbs and the body segment that includes at least the first limb and the second limb from the bioimpedance measurements.

The control unit can be configured to normalise the measures of extracellular fluid and/or intracellular fluid in the first and second limbs using the bioimpedance measurement for the body segment between that includes at least the first limb and the second limb.

Alternatively the control unit can be configured to determine the ratio of extracellular fluid to intracellular fluid in each of the first and second limbs and the body segment that includes at least the first limb and the second limb.

In some embodiments the control unit is configured to normalise the ratio of extracellular fluid to intracellular fluid for each of the first and second limbs using the ratio of extracellular fluid to intracellular fluid for the body segment that includes at least the first limb and the second limb.

In some embodiments the control unit is configured to obtain measurements of the bioimpedance of the first limb, second limb and the segment of the body that includes at least the first limb and the second limb for an alternating current at a single frequency. The alternating current at a single frequency can be at a low frequency, and preferably, the low frequency is a frequency at or around 10 kHz.

However, in preferred embodiments, the control unit is configured to obtain the measurements of the bioimpedance of the first limb, second limb and the segment of the body that includes at least the first limb and the second limb for alternating currents at first and second frequencies, wherein the first frequency is lower than the second frequency.

The first (low) frequency can be a frequency at or around 10 kHz and the second (high) frequency can be a frequency at or around 1 MHz.

In alternative embodiments the control unit is configured to obtain the measurements of the bioimpedance of the first limb, second limb and the segment of the body that includes at least the first limb and the second limb for alternating currents at a plurality of frequencies. Preferably, each of the plurality of frequencies are in the range of 5 kHz to 1 MHz.

In some embodiments the apparatus further comprises first and second current electrodes that are configured to be attached to the first limb and the second limb of the subject respectively; a first set of measurement electrodes that are configured to be attached to the first limb; and a second set of measurement electrodes that are configured to be attached to the second limb.

In some embodiments, the control unit is configured to obtain the measurement of the bioimpedance of the first limb of the subject using the first and second current electrodes and the first set of measurement electrodes; obtain the measurement of the bioimpedance of the second limb of the subject using the first and second current electrodes and the second set of measurement electrodes; and obtain the measurement of the bioimpedance of the segment of the body that includes at least the first limb and the second limb using the first and second current electrodes and one of the measurement electrodes in the first set of measurement electrodes and one of the measurement electrodes in the second set of measurement electrodes.

In some embodiments the apparatus further comprises a current source that is connected to the current electrodes and that is configured to selectively output an alternating current at one or more frequencies.

In some embodiments the first and second limbs are the legs of the subject and the segment of the body that includes at least the first limb and the second limb is the lower body of the subject.

In other embodiments the first and second limbs are the arms of the subject and the segment of the body that includes at least the first limb and the second limb is the upper body of the subject.

In some embodiments the apparatus further comprises a first structure configured to be attached to the first limb, the first structure having embedded or arranged therein the first current electrode and the first set of measurement electrodes; a second structure configured to be attached to the second limb, the second structure having embedded or arranged therein the second current electrode and the second set of measurement electrodes; wherein the first and second structures are such that the respective electrodes are in a fixed relationship with each other.

According to a second specific aspect of the invention, there is provided a method of estimating the fluid content in a subject, the method comprising obtaining a measurement of the bioimpedance of a first limb of the subject; obtaining a measurement of the bioimpedance of a second limb of the subject; obtaining a measurement of the bioimpedance of a segment of the body that includes at least the first limb and the second limb; and determining a measure of the fluid content in the first limb using the bioimpedance measurement of the first limb, the bioimpedance measurement of the second limb and the bioimpedance measurement of the segment of the body that includes at least the first limb and the second limb.

In some embodiments the step of determining a measure of the fluid content in the first limb comprises normalising the bioimpedance measurements for the first and second limbs using the bioimpedance measurement for the body segment that includes at least the first limb and the second limb.

In other embodiments the step of determining a measure of the fluid content in the first limb comprises determining the amount of extracellular fluid and/or intracellular fluid in the first and second limbs and the body segment that includes at least the first limb and the second limb from the bioimpedance measurements.

In some embodiments the step of determining a measure of the fluid content in the first limb comprises normalising the measures of extracellular fluid and/or intracellular fluid in the first and second limbs using the bioimpedance measurement for the body segment that includes at least the first limb and the second limb.

In some embodiments the step of determining a measure of the fluid content in the first limb comprises determining the ratio of extracellular fluid to intracellular fluid in each of the first and second limbs and the body segment that includes at least the first limb and the second limb.

In some embodiments the step of determining a measure of the fluid content in the first limb comprises normalising the ratio of extracellular fluid to intracellular fluid for each of the first and second limbs using the ratio of extracellular fluid to intracellular fluid for the body segment that includes at least the first limb and the second limb.

In some embodiments the steps of obtaining comprise obtaining the measurements of the bioimpedance of the first limb, second limb and the segment of the body that includes at least the first limb and the second limb for an alternating current at a single frequency. In these embodiments the alternating current is preferably at a low frequency. The low frequency is preferably a frequency at or around 10 kHz.

In preferred embodiments the steps of obtaining comprise obtaining the measurements of the bioimpedance of the first limb, second limb and the segment of the body that includes at least the first limb and the second limb for alternating currents at first and second frequencies, wherein the first frequency is lower than the second frequency. Preferably the first (low) frequency is a frequency at or around 10 kHz and the second (high) frequency is a frequency at or around 1 MHz.

In other embodiments the steps of obtaining comprise obtaining the measurements of the bioimpedance of the first limb, second limb and the segment of the body that includes at least the first limb and the second limb for alternating currents at a plurality of frequencies. Preferably each of the plurality of frequencies are in the range of 5 kHz to 1 MHz.

In some embodiments the first and second limbs are the legs of the subject and the segment of the body that includes at least the first limb and the second limb is the lower body of the subject.

In other embodiments the first and second limbs are the arms of the subject and the segment of the body that includes at least the first limb and the second limb is the upper body of the subject.

In some embodiments the step of obtaining a measurement of the bioimpedance of the first limb of the subject comprises obtaining the measurement using first and second current electrodes that are attached to a first limb and a second limb of the subject respectively and a first set of measurement electrodes that are attached to the first limb; the step of obtaining a measurement of the bioimpedance of the second limb of the subject comprises obtaining the measurement using the first and second current electrodes and a second set of measurement electrodes that are attached to the second limb; and the step of obtaining a measurement of the bioimpedance of the body segment that includes at least the first limb and the second limb comprises obtaining the measurement using the first and second current electrodes, one of the measurement electrodes in the first set of measurement electrodes and one of the measurement electrodes in the second set of measurement electrodes.

According to a third aspect of the invention, there is provided a computer program product having computer readable code embodied therein, the computer readable code being configured such that, on execution by a suitable computer or processing unit, the computer or processing unit is caused to perform any of the methods described above.

### BRIEF DESCRIPTION OF THE DRAWINGS

Exemplary embodiments of the invention will now be described, by way of example only, with reference to the following drawings, in which:

FIG. 1 is a block diagram of an apparatus according to an aspect of the invention;

FIG. 2 illustrates the apparatus of FIG. 1 attached to a subject to enable bioimpedance measurements of the legs of the subject;

FIG. 3 is a flow chart illustrating a method of measuring the fluid content in a subject according to an aspect of the invention;

FIG. 4 illustrates the use of the Cole-Cole model to determine intracellular fluid and extracellular fluid in a body segment from bioimpedance measurements;

FIG. 5 is a flow chart illustrating a method of measuring the extracellular fluid in a subject according to a first specific embodiment of the invention;

FIG. 6 is a flow chart illustrating a method of measuring the extracellular fluid in a subject according to a second specific embodiment of the invention;

FIG. 7 is a flow chart illustrating a method of measuring the fluid in a subject according to a third specific embodiment of the invention; and

FIG. 8 is a diagram illustrating electrode strips that can form part of the apparatus according to a specific embodiment of the invention.

### DETAILED DESCRIPTION OF THE PREFERRED EMBODIMENTS

As described above, the invention provides that, rather than obtain bioimpedance measurements of separate non-overlapping body segments, bioimpedance measurements are made for ‘overlapping’ body segments, and the measurements are used to assess the extracellular fluid content in the body segments. The segments are overlapping in the sense that one of the segments includes another one of the segments. For example, separate bioimpedance measurements can be made for each leg and for the whole lower body of the subject (e.g. by measuring the bioimpedance from the left foot to the right foot). The lower body segment thus overlaps the left leg and right leg segments. Other overlapping segments can comprise each arm of the subject and the upper body of the subject, or each of the left/right limbs and the left/right side of the body (which includes both of the left/right limbs).

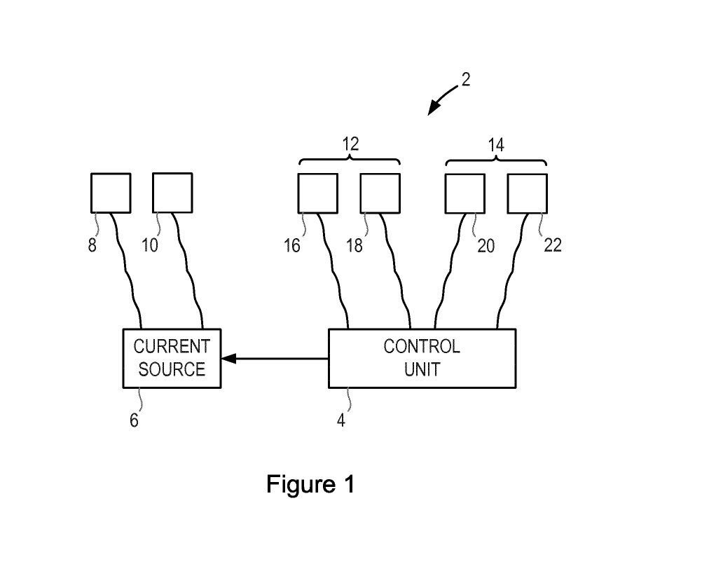

FIG. 1 illustrates an apparatus 2 for measuring the fluid content of part of the body of a subject according to an embodiment of the invention. The apparatus 2 is typically constructed in a form that can be easily worn by or attached to a subject in a clinical or home setting. The apparatus 2 comprises a control unit 4 that is configured to control the operation of the apparatus 2, including the application of electrical current to the subject, determining the bioimpedance from the voltages measured from the various parts of the body of the subject and determining a measure of the extracellular fluid (or more specifically edema formation) in the or a part of the body of the subject. The control unit 4 may be configured according to the invention using hardware, software, firmware or a combination thereof. The control unit 4 can take the form of a small dedicated processing device, a smart phone, a laptop computer, a desktop computer or any other suitable type of processing device.

The control unit 4 is connected to a current source 6 that is configured to output an alternating electrical current having at least one frequency under the control of the control unit 4. The at least one frequency can include a relatively low frequency, for example in the range of 3-15 kHz, such as a frequency at or around 10 kHz, although outputting electrical currents at other frequencies is possible. In preferred embodiments, the current source 6 is configured to selectively output an alternating electrical current at different (i.e. two or more discrete) frequencies under the control of the control unit 4. In some embodiments, the current source 6 is configured to selectively output electrical currents of at least two discrete frequencies in the range of 5 kHz to 1 MHz, and preferably at least a relatively low frequency in the range 3-15 kHz and a relatively high frequency in the range 500-1000 kHz. In some embodiments, the current source 6 is configured to selectively output an electrical current having a frequency of 5 kHz or 10 kHz and an electrical current having a frequency of 1 MHz.

Two electrodes 8, 10 are provided that are connected to the current source 6, and that are used to apply or inject the alternating current to the subject. The electrodes 8, 10 are therefore suitable for attachment to the skin of a subject, and can be of any suitable construction to enable a good and consistent electrical contact to the skin. According to the invention, the electrodes 8, 10, which are also referred to herein as ‘current’ electrodes or ‘current-injecting’ electrodes (i.e. the electrodes used for injecting current into the subject), are to be attached to respective limbs of the subject (e.g. each arm, each leg, or an arm and a leg) so as to pass the alternating electrical current through a segment of the body of the subject that includes at least the respective limbs. In preferred implementations, the electrodes 8, 10 are configured to be attached to a finger or toe of the subject, to the palm or back of a hand of the subject, to the wrist or ankle of the subject, or to the sole or top of a foot of the subject.

The apparatus 2 also comprises two pairs or sets of electrodes 12, 14 that are connected to the control unit 4 and that are used to measure the voltage (or differential potential) across different parts of the body (a ‘body segment’) of the subject. The first pair or set of electrodes 12 comprises two measurement electrodes 16, 18 (i.e. electrodes used to make measurements of the voltage) and the second pair or set of electrodes 14 comprises a further two electrodes 20, 22. As with the current injecting electrodes 8, 10, the electrodes 16, 18, 20, 22 are suitable for attachment to the skin of a subject, and can be of any suitable construction to enable a good and consistent electrical contact to the skin. The first and second pairs of electrodes 12, 14 are to be attached to respective limbs of the subject (corresponding to the limbs to which the current-injecting electrodes 8, 10 are attached) to enable a voltage measurement to be made for that limb.

As noted above, the control unit 4 is connected to the measurement electrodes 16, 18, 20, 22 and uses the voltage measurements obtained using the electrodes to determine the bioimpedance of the limbs to which the pairs of measurement electrodes 12, 14 are attached. As well as using the measurement electrodes 16, 18, 20, 22 to measure the bioimpedance of the limbs to which the pairs of measurements electrodes are attached, the control unit 4 is also configured to measure the voltage across the larger (overlapping) body segment between the current electrodes 8, 10. That is, the control unit 4 measures the voltage between one of the electrodes 16, 18 in the first pair of electrodes 12 and one of the electrodes 20, 22 in the second pair of electrodes 14 (as well as the voltage between the electrodes 16, 18 in the first pair of electrodes 12 and the voltage between the electrodes 20, 22 in the second pair of electrodes 14). The bioimpedance measurement obtained in this way corresponds to the bioimpedance of the body segment that includes the two limbs to which the current electrodes 8, 10 are attached and any intervening tissue (e.g. the chest in the case of the current electrodes 8, 10 being attached to the arms, or the waist in the case of the current electrodes 8, 10 being attached to the legs). The control unit 4 is configured to determine the complex bioimpedance Z of the body segment (e.g. limb, upper body, lower body, etc.) using Ohm's law: Z=U/I, where U is the measured voltage and I is the applied current. The way in which the bioimpedance Z is measured for a body segment is generally conventional, and those skilled in the art will be aware of how to perform the bioimpedance measurement, including measuring the real and imaginary part of the voltage drop between the electrodes by introducing a phase shift. Those skilled in the art will also be aware of various pre-processing steps that can be performed on the measured voltage in order to obtain an accurate measure of the bioimpedance, and such steps will not be described herein.

It will be appreciated that in some embodiments, the current electrodes 8, 10 and/or the measurement electrodes 16, 18, 20, 22 can be integrated into an item of clothing (e.g. sock, stocking, glove, jumper, etc.) that is to be worn by the subject.

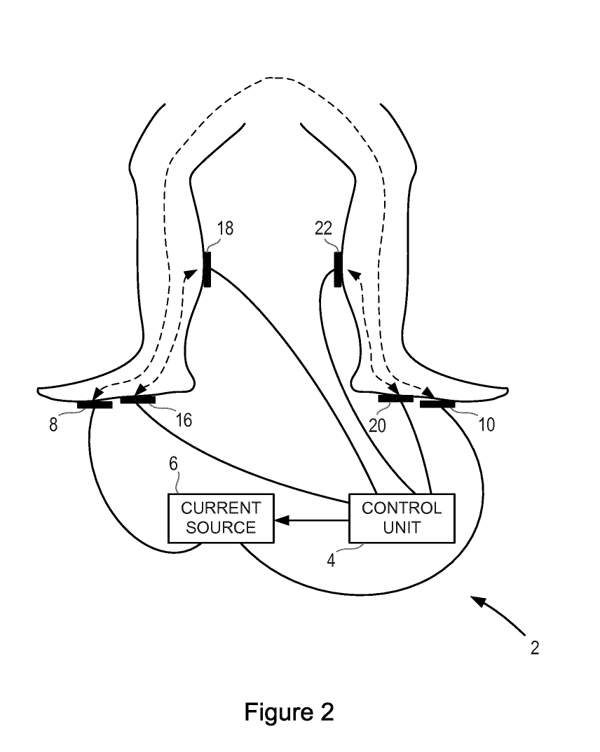

FIG. 2 illustrates the apparatus of FIG. 1 attached to a subject to enable bioimpedance measurements of the legs of the subject. Thus, one of the current electrodes 8 is attached to or otherwise in contact with the skin of the right foot of the subject and the other one of the current electrodes 10 is attached to or otherwise in contact with the skin of the left foot of the subject. The first pair of measurement electrodes 12 are attached to the right foot (electrode 16) and right calf (electrode 18) of the subject to enable a measurement to be made of the voltage in the right leg, and the second pair of measurement electrodes 14 are attached to the left foot (electrode 20) and left calf (electrode 22) of the subject to enable a measurement to be made of the voltage in the left leg. It will be appreciated that the apparatus 2 could instead be used to make bioimpedance measurements of the arms of the subject, in which case the electrodes 8, 16 would be attached to the right hand, electrode 18 would be attached to the right forearm or right upper arm, electrodes 10, 20 would be attached to the left hand, and electrodes 22 would be attached to the left forearm or left upper arm.

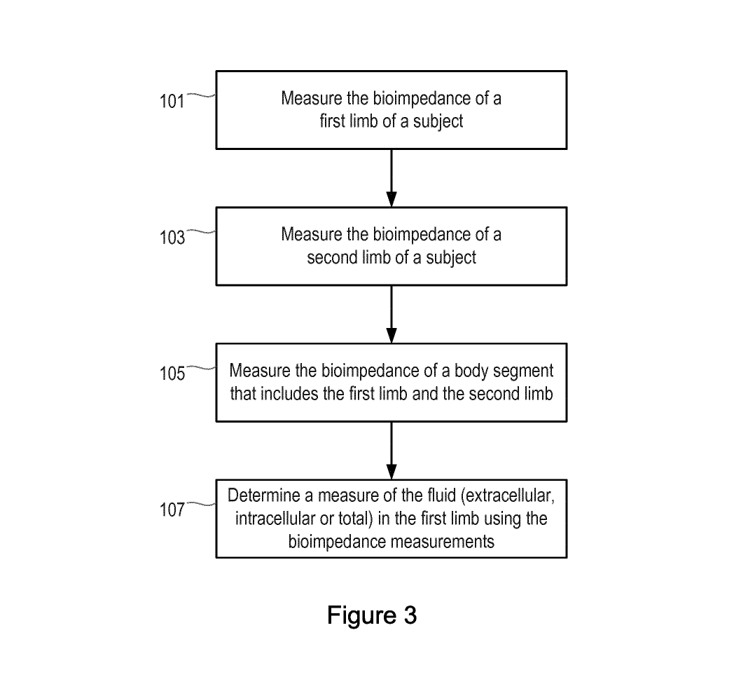

The flow chart in FIG. 3 illustrates a method of measuring the fluid content in a subject according to an aspect of the invention. In preferred embodiments, the method in FIG. 3 is performed using the apparatus 2 described above.

In a first step, step 101, which is performed after the electrodes 8, 10, 16, 18, 20, 22 have been attached to the subject, the bioimpedance of a first limb of the subject is measured. The measurement is made by the control unit 4 controlling the current source 6 to output an alternating current at a particular frequency (e.g. in the range 5 kHz to 1 MHz) through the electrodes 8, 10 and the control unit 4 measures the voltage in the first limb using the first pair of measurement electrodes 12. In some embodiments, the measurement is repeated at least once using an alternating current at a different frequency to enable the extracellular fluid and intracellular fluid in the first limb to be separately determined. In other embodiments, only the total fluid content of the first limb is determined. As noted above, the control unit 4 determines the bioimpedance measurement from the voltage U using Z=U/I.

In step 103 the bioimpedance of a second limb of the subject is measured. This measurement is performed in a similar way to the bioimpedance measurement of the first limb (e.g. with an alternating current at the same frequency or frequencies) using the second pair of measurement electrodes 14 that are attached to the second limb.

In step 105, the bioimpedance of the body segment that overlaps the first limb and second limb is measured. The measurement of the bioimpedance of the overlapping body segment is performed in a similar way to the measurement of the bioimpedance of the first limb, using an alternating current at the same frequency or frequencies. The measurement is performed using one of the electrodes 16, 18 in the first pair of electrodes 12 and one of the electrodes 20, 22 in the second pair of electrodes 14. Preferably, measurement is performed using the measurement electrodes that are located closest to the current electrode 8, 10. For example, in the embodiment shown in FIG. 2, the bioimpedance measurement of the lower body segment is preferably made using measurement electrodes 16 and 20.

Then, in step 107, a measure of the fluid content in the first limb is determined using the bioimpedance measurements. This step may also comprise determining a measure of the fluid content in the second limb and/or the body segment comprising the first limb and second limb. The bioimpedance measurements can be used in a number of different ways to determine this measure. In some embodiments, the bioimpedance measurements for the first limb and the second limb can each be ‘normalised’ by dividing by the bioimpedance measurement for the overlapping segment (i.e. the body segment extending from the first limb to the second limb) and the normalised bioimpedance measurements can be compared. This way, the amount of fluid (extracellular, intracellular or total) in a limb is expressed as a proportion of the amount of fluid (extracellular, intracellular or total) in the overlapping body segment (which includes the limb, the other limb and the intervening tissue). Since peripheral edema mainly forms in the outer extremities (i.e. in the lower legs (e.g. calves) rather than the upper legs (e.g. thighs), and in the forearms rather than the upper arms), this normalisation corrects both for inter-measurement variability and inter-patient variability.

In other embodiments, the bioimpedance measurements are processed to determine the amount of fluid (and preferably separately determine the amounts of intracellular fluid and extracellular fluid) in the body segments (e.g. first limb, second limb and overlapping segment), and these amounts are used to determine a measure of the extracellular fluid in the first limb. In these embodiments, the determined amount of extracellular fluid in the first limb and the second limb can each be ‘normalised’ by dividing by the determined amount of extracellular fluid in the overlapping body segment. In these and other embodiments, a ratio of the extracellular fluid and the intracellular fluid can be determined for each body segment and those ratios combined and/or compared.

It will be appreciated that step 107 does not have to be performed straight after the bioimpedance measurements are obtained in steps 101, 103 and 105. In addition, although steps 101, 103 and 105 indicate that the steps include the making of the relevant measurement of the bioimpedance, it will be appreciated that steps 101, 103 and 105 can alternatively comprise retrieving a previously obtained measurement of bioimpedance from a memory module.

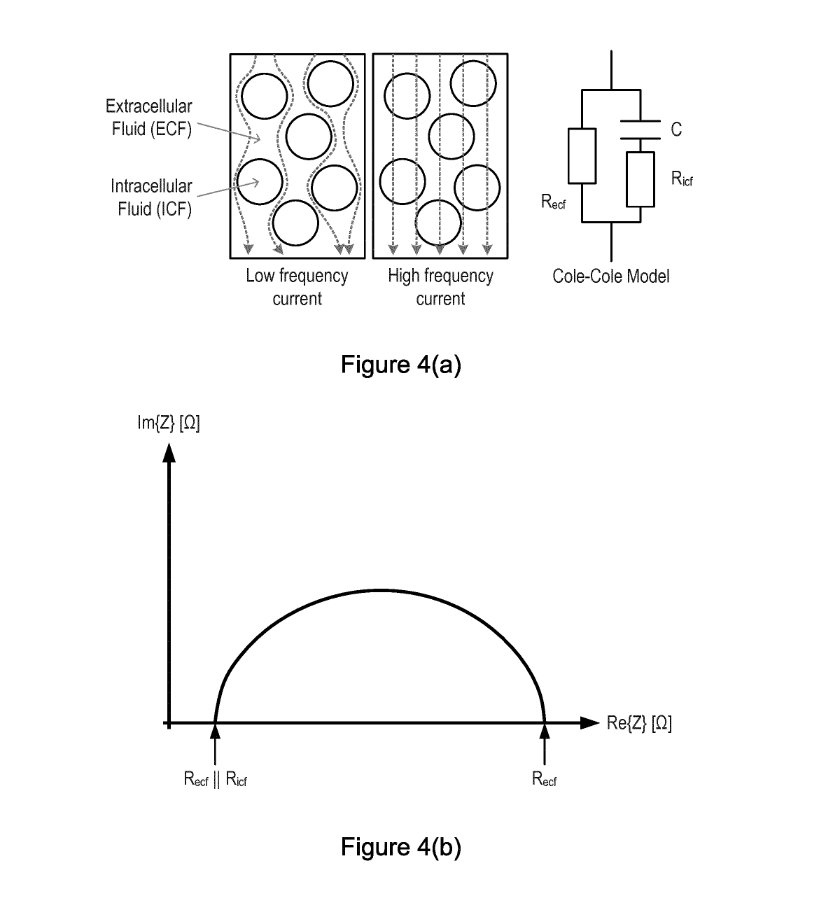

The way in which the apparatus 2 is operated and the bioimpedance measurements processed to determine the amounts fluid (and preferably the amounts of intracellular fluid and extracellular fluid) in the body segments is described in more detail below with reference to FIG. 4.

If bioimpedance measurements are obtained using electrical current at a single (low) frequency, then it is possible to determine a measure of the total amount of fluid (i.e. the extracellular fluid and the intracellular fluid combined) in the relevant body segment.

However, obtaining multiple bioimpedance measurements using currents having different frequencies allows the resistance of extracellular water and intracellular water to be separately determined, as discussed in more detail below.

FIG. 4(a) illustrates the flow of current through tissue at high and low frequencies and an equivalent electrical circuit model of the tissue. At low measurement frequencies (e.g. approaching 0 Hz—direct current) the measured biological tissue impedance is mainly determined by the extracellular fluid content and its characteristics. At these low frequencies, the injected current does not easily pass through cell membranes (shown by the dashed arrows in FIG. 4(a)) since they have a capacitive behaviour), and thus the capacitor acts as an open circuit and current only flows through the extracellular fluid, which has resistance R_{ecf}. At higher frequencies the electrical properties of the biological tissue are determined by both the intracellular and extracellular fluid content as the injected current is able to pass through the cell membranes (shown by the solid arrows in FIG. 4(a)). In the circuit model, as the frequency increases towards infinity, the capacitor Cₘ acts as a short circuit and the current will flow through both the intracellular fluid (with resistance R_{icf}) and extracellular fluid (with resistance R_{ecf}). Therefore, the influence of the intra- and extra-cellular fluid content on the measured bioimpedance depends on the frequency of the injected current. This allows a characterization of the electrical properties of the biological tissue according to the Cole-Cole model, which is shown in FIG. 4(b). Thus, in some embodiments, by taking bioimpedance measurements at multiple (at least two, but preferably four or more) frequencies, an approximation by interpolation of the electrical properties of the tissue at direct current (DC, frequency of zero Hz) when the extracellular fluid content is the main component of the impedance can be made. The resistance of the extracellular fluid is denoted R_{ecf} and is equal to R₀ (i.e. the resistance measured with a direct current), and the resistance of the intracellular fluid is denoted R_{icf}. The resistance that would be measured at an infinite frequency is denoted R_{inf} and is a function of R_{ecf} and R_{icf}. In particular, R_{inf}=R_{ecf}∥R_{icf}=>R_{icf}=R_{inf}R_{ecf}/(R_{ecf}−R_{inf}). Thus, with a measurement made at a low frequency the extracellular resistance (R₀=R_{ecf}) can be determined and then R_{icf} can be computed from the measurement at a high frequency. Those skilled in the art will be aware of suitable techniques for performing this interpolation of the bioimpedance measurements obtained at multiple frequencies to obtain measures of the extracellular and intracellular fluid.

In alternative embodiments where measurements of bioimpedance are taken using current at a single frequency, it is not possible to separately determine the components of intra- and extra-cellular fluid content in the measured bioimpedance. Instead, the bioimpedance measurement is used to determine the total fluid content of the relevant body segment.

Determining a measure of the extracellular fluid using the apparatus of FIG. 1 and method of FIG. 3 provides several advantages of the conventional methods. In particular, the apparatus 2 has good versatility, minimal complexity and improved reproducibility and reliability compared to the conventional techniques (not least because the three bioimpedance measurements are taken using only two current electrodes and two pairs of measurement electrodes that do not need to be repositioned for the different measurements).

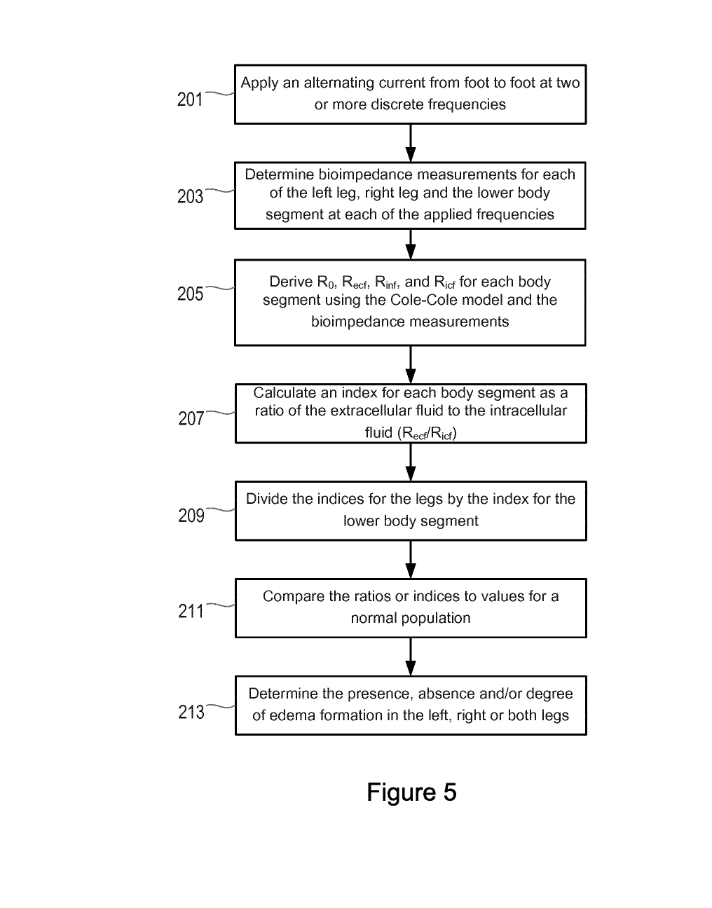

A method of measuring the extracellular fluid in a subject according to a first specific embodiment is shown in FIG. 5. It will be appreciated that although this embodiment is described with reference to identifying tissue edema in the lower legs, it can equally be applied to identifying tissue edema in the arms. In a first step, step 201, an alternating current is applied from foot to foot at two or more discrete frequencies in the range of 5 kHz to 1 MHz. Bioimpedance measurements are determined for each of the left leg, right leg and the lower body segment (which includes the left leg and right leg) at each of the applied frequencies (step 203).

Next, in step 205, R₀, R_{ecf}, R_{inf}, and R_{icf} are derived for each body segment using the Cole-Cole model for bioimpedance spectroscopy and the multiple bioimpedance measurements for each body segment.

In step 207, a ratio of the extracellular fluid to the intracellular fluid (R_{ecf}/R_{icf}) is calculated for each body segment to give an index for each body segment. This gives r_{leftleg}=R_{leftleg}_{_}_{ecf}/R_{leftleg}_{_}_{icf}, r_{rightleg}=R_{rightleg}_{_}_{ecf}/R_{rightleg}_{_}_{icf}, and r_{lowerbody}=R_{lowerbody}_{_}_{ecf}/R_{lowerbody}_{_}_{icf}.

Then, in step 209, the index for the left leg is divided by the index for the lower body (r_{leftleg}/r_{lowerbody}) to give a measure of the proportion of extracellular water in the left leg. Similarly, the index for the right leg is divided by the index for the lower body (r_{rightleg}/r_{lowerbody}) to give a measure of the proportion of extracellular water in the right leg. A further ratio can be calculated as the sum of the indices for the left and right legs over the index for the lower body segment (i.e. (r_{leftleg}+r_{rightleg})/r_{lowerbody})

In step 211 one or more of the ratios or indices determined in steps 207 and 209 are compared to each other and/or compared to ratios obtained in a normal population of preferably similar subjects (e.g. similar in sex, age and body mass index (BMI) and/or compared to values obtained in previous measurements in the same subject.

Finally, in step 213, the presence, absence and/or degree of edema formation in the left leg, right leg and/or both legs is determined or estimated based on the result of the comparison in step 211.

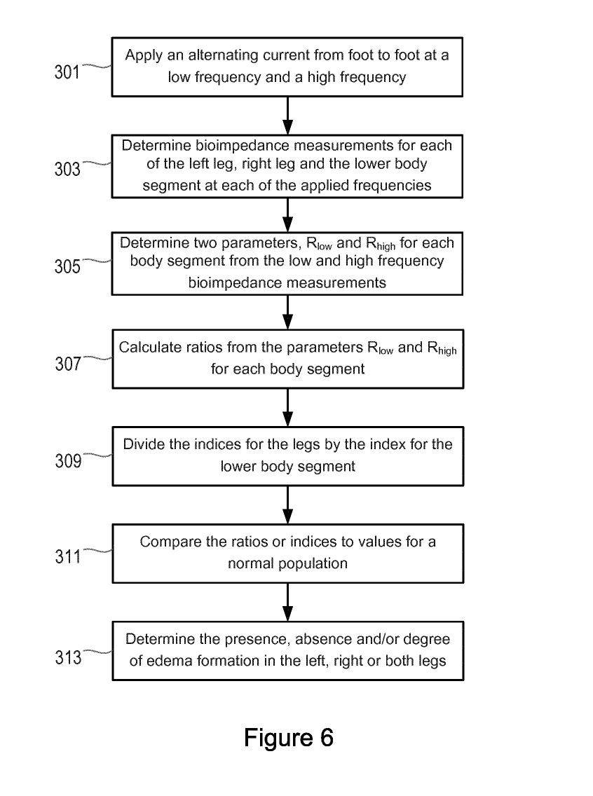

A method of measuring the extracellular fluid in a subject according to a second specific embodiment is shown in FIG. 6. As with the first specific embodiment described above, it will be appreciated that although this embodiment is described with reference to identifying tissue edema in the lower legs, it can equally be applied to identifying tissue edema in the arms. In a first step, step 301, an alternating current is applied from foot to foot at two discrete frequencies in the range of 5 kHz to 1 MHz. Preferably one of the frequencies is at the lower end of the frequency range (e.g. 10 kHz) and the other frequency is at the higher end of the frequency range (e.g. 1 MHz). Bioimpedance measurements are determined for each of the left leg, right leg and the lower body segment (which includes the left leg and right leg) at both of the applied frequencies (step 303).

Next, in step 305, the low and high frequency bioimpedance measurements are used to determine two parameters, R_{low} and R_{high} for each body segment. R_{low} and R_{high} for a body segment can be calculated as a function of Z_{low} (the impedance measured at the low frequency) and Z_{high} (the impedance measured at the high frequency). For example, R_{low}=|Z_{low}|, the absolute value of Z_{low}, or R_{low}=Re{Z_{low}} the real part of Z_{low}. Likewise, R_{high}=|Z_{high}| or R_{high}=Re{Z_{high}}. Those skilled in the art will be aware of other functions that could be used to obtain values for R_{low} and R_{high} and thus approximate the bioimpedance for the extracellular fluid and intracellular fluid respectively.

In step 307, various ratios are calculated from the parameters R_{low} and R_{high} for each body segment. In particular, a ratio of the extracellular fluid to the intracellular fluid (R_{low}/R_{high}) is calculated for each body segment to give an index for each body segment. This gives r_{leftleg}=R_{leftleg}_{_}_{low}/R_{leftleg}_{_}_{high}, r_{rightleg}=R_{rightleg}_{_}_{low}/R_{rightleg}_{_}_{high}, and r_{lowerbody}=R_{lowerbody}_{_}_{low}/R_{lowerbody}_{_}_{high}.

Then, in step 309 the index for the left leg is divided by the index for the lower body (r_{leftleg}/r_{lowerbody}) to give a measure of the proportion of extracellular water in the left leg. Similarly, the index for the right leg is divided by the index for the lower body (r_{leftleg}/r_{lowerbody}) to give a measure of the proportion of extracellular water in the right leg. A further ratio can be calculated from the sum of the indices for the left and right legs over the index for the lower body segment (i.e. (r_{leftleg}+r_{rightleg})/r_{lowerbody}).

In step 311 one or more of the ratios or indices determined in steps 307 and 309 are compared to each other and/or compared to ratios obtained in a normal population of preferably similar subjects (e.g. similar in sex, age and body mass index (BMI) and/or compared to values obtained in previous measurements in the same subject.

Finally, in step 313, the presence, absence and/or degree of edema formation in the left leg, right leg and/or both legs is determined or estimated based on the result of the comparison in step 311.

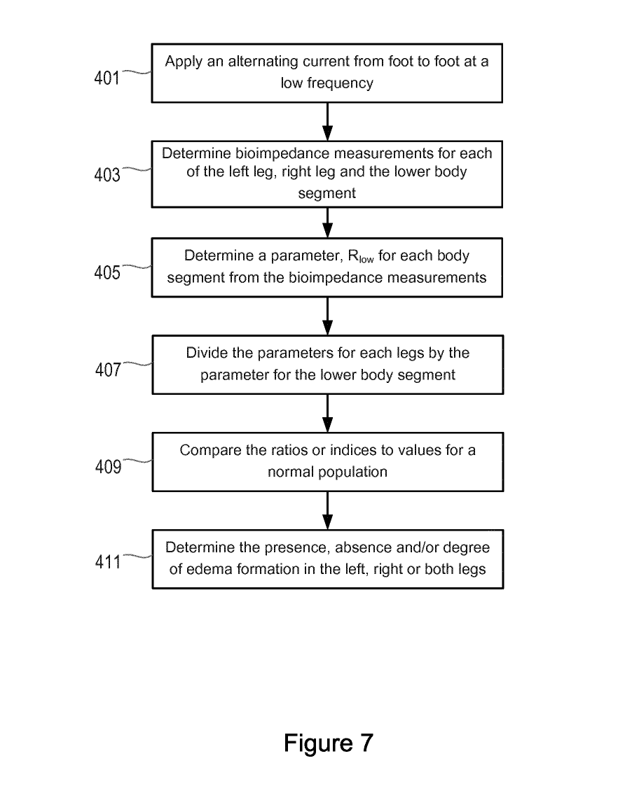

A method of measuring the extracellular fluid in a subject according to a third specific embodiment is shown in FIG. 7. As with the first and second specific embodiments described above, it will be appreciated that although this embodiment is described with reference to identifying tissue edema in the lower legs, it can equally be applied to identifying tissue edema in the arms. In a first step, step 401, an alternating current is applied from foot to foot at a single low frequency (i.e. a low frequency in the range of 5 kHz to 1 MHz, for example 10 kHz). Bioimpedance measurements are determined for each of the left leg, right leg and the lower body segment (which includes the left leg and right leg) at the applied frequency (step 403). It will be appreciated that as the bioimpedance is only measured at one frequency, it is not possible to derive separate measures of the amount of extracellular fluid and intracellular fluid in the body segment. Instead, the bioimpedance measurement is used as an indication of the total fluid content of the body segment.

Thus, in step 405, the bioimpedance measurement is used to determine a parameter, R_{low}, for each body segment. R_{low} for a body segment can be calculated as a function of Z_{low} (the impedance measured at the low frequency). For example, R_{low}=|Z_{low}|, the absolute value of Z_{low} or R_{low}=Re{Z_{low}}, the real part of Z_{low}.

In step 407, various ratios are calculated from the parameters R_{low} for each body segment. In particular, a ratio of R_{low} for the left leg and the lower body is calculated, a ratio of R_{low} for the right leg and the lower body is calculated, and a ratio of the sum of the value of R_{low} for the left and right legs over R_{low} for the lower body segment is calculated.

Then, in step 409 one or more of the ratios determined in step 407 are compared to each other and/or compared to ratios obtained in a normal population of preferably similar subjects (e.g. similar in sex, age and body mass index (BMI) and/or compared to values obtained in previous measurements in the same subject.

Finally, in step 411, the presence, absence and/or degree of edema formation in the left leg, right leg and/or both legs is determined or estimated based on the result of the comparison in step 409.

In a further embodiment of the invention, the apparatus 2 can be integrated in another type of apparatus used to measure a physiological characteristic of a subject. For example, the apparatus 2 according to the invention could be incorporated into the foot patches on a set of weighing scales, which would allow measurements of bioimpedance and weight to be obtained using a single apparatus.

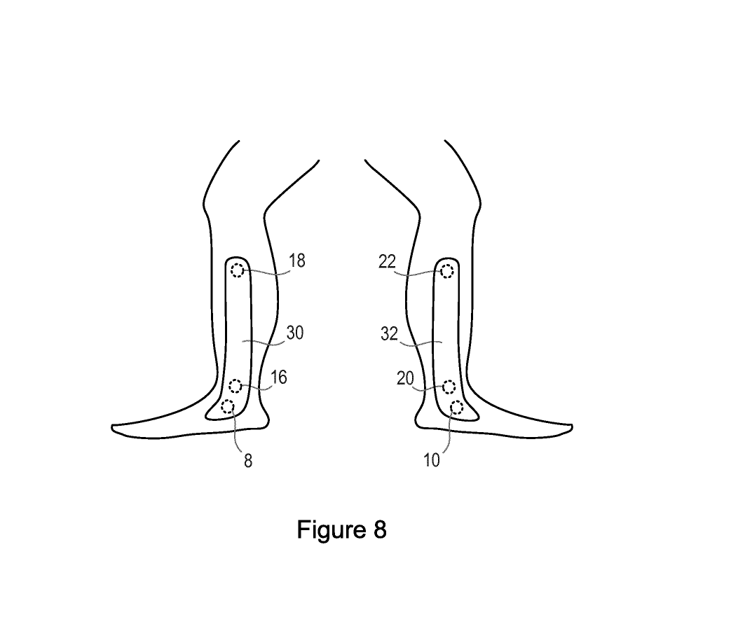

A further advantageous embodiment of the invention is illustrated in FIG. 8. In this embodiment the electrodes 8, 10, 16, 18, 20, 22 are embedded in or arranged in a structure 30, 32 that holds the electrodes for each limb in a fixed arrangement with respect to each other and thus enables the electrodes to be consistently attached to the subject at the same locations to minimize measurement errors due to inconsistencies in electrode placement. For example, as shown in FIG. 8, the electrodes 8, 16, 20 and 10, 18, 22 could be embedded in respective strips 30, 32 (of approximately 30 cm in length) which, for example, are shaped to receive the bottom of a foot and extend up to the calf or be otherwise attached to a leg. Alternatively, the strips 30, 32 can be shaped to attach to the upper side of the hands and the forearms. It will be appreciated that these strips 30, 32 could be embedded in socks, stockings, gloves, sleeves, etc. for improved repeatability. In view of the above teaching, those skilled in the art will readily contemplate other types or forms of structure or device 30, 32 in which the electrodes could be fixed in order to improve the repeatability of bioimpedance measurements.

In a variation of the embodiment shown in FIG. 8, a device can be provided that is configured to receive both of the subject's feet (or arms) and that comprises the required electrodes in a fixed arrangement. The device is arranged such that the subject's feet (or arms) only fit into the device in one particular position, which means that consistency in electrode placement/attachment can be further improved over the embodiments shown in FIG. 8.

Various applications of the invention are contemplated. Some of these applications or uses of the invention are described below.

Edema formation at home—Several home care patient populations are at risk of developing peripheral edema, including patients with heart failure, nephrotic syndrome, liver cirrhosis, diabetes, hypertension and patients who had lymph surgery (e.g. as part of breast cancer surgery). Furthermore, pregnancies are often complicated by hypertension which also results in peripheral edema. A device or apparatus that measures tissue water content would provide an early warning for edema formation in these patient populations.

Provide guidance for fluid therapy in hospitalized patients—Hypovolemia is a common problem in many hospitalized patients and the first treatment of choice is fluid therapy. However, since fluids sometime leak out of the vasculature, large volumes are required, potentially leading to a fluid overload. Fluid overload is common in the ward, intensive care unit (ICU), and operating room (OR). Specific patient populations particularly at risk of fluid overload are septic patients due to leaky vasculature, patients with renal dysfunction, and patients undergoing heart surgery requiring a heart-lung machine (e.g., coronary artery bypass graft (CABG) surgery). A device or apparatus measuring peripheral edema would provide an early warning for fluid overload in patients receiving intravenous fluids since the first sign of fluid overload is peripheral tissue edema formation.

Provide guidance for haemodialysis—Patients with end stage kidney disease receive hemodialysis multiple times per week. However, the dialysis end-point or target is very ill-defined (i.e., only weight-based). A device or apparatus measuring peripheral tissue water content would provide guidance for hemodialysis for patients with chronic kidney disease.

Early warning for dehydration—Dehydration is a big issue amongst many home care patient populations, including babies, children in developed and developing countries, elderly, pregnant women, diabetics, and athletes, mountaineers, and soldiers. Especially in the neonatal and pediatric intensive care units, dehydration is a major problem which develops very rapidly. A device or apparatus that reliably and reproducibly measures peripheral tissue water content would provide an early warning for dehydration in these people/patients.

Rehabilitation—A device or apparatus that measures peripheral tissue water content and thereby indicates the muscle volume could aid rehabilitation after injury or surgery by comparing the muscle volume in the injured limb with the muscle volume in the healthy limb.

There is therefore provided an improved method and apparatus for estimating the extracellular fluid content of part of the body of a subject that has good versatility, minimal complexity and improved reproducibility and reliability compared to conventional techniques.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus for estimating the fluid content in a subject, the apparatus comprising:
a control unit that is configured to:
obtain a measurement of the bioimpedance of a first limb of the subject;
obtain a measurement of the bioimpedance of a second limb of the subject;
obtain a measurement of the bioimpedance of a segment of the body that includes at least the first limb and the second limb; and
determine a measure of the fluid content in the first limb using the bioimpedance measurement of the first limb, the bioimpedance measurement of the second limb and the bioimpedance measurement of the segment of the body that includes at least the first limb and the second limb.

2. An apparatus as claimed in claim 1, wherein the control unit is configured to determine a measure of the fluid content in the first limb by normalising the bioimpedance measurements for the first and second limbs using the bioimpedance measurement for the body segment that includes at least the first limb and the second limb.

3. An apparatus as claimed in claim 1, wherein the control unit is configured to determine a measure of the fluid content in the first limb by determining the amount of extracellular fluid and/or intracellular fluid in the first and second limbs and the body segment that includes at least the first limb and the second limb from the bioimpedance measurements, and normalising the measures of extracellular fluid and/or intracellular fluid in the first and second limbs using the measure of extracellular and/or intracellular fluid in the body segment that includes at least the first limb and the second limb.

4. An apparatus as claimed in claim 1, wherein the control unit is configured to determine a measure of the fluid content in the first limb by determining a ratio of extracellular fluid to intracellular fluid in each of the first and second limbs and the body segment that includes at least the first limb and the second limb from the bioimpedance measurements, and normalise the ratio of extracellular fluid to intracellular fluid for each of the first and second limbs using the ratio of extracellular fluid to intracellular fluid for the body segment that includes at least the first limb and the second limb.

5. An apparatus as claimed in any of claims 1-4, wherein the control unit is configured to obtain measurements of the bioimpedance of the first limb, second limb and the segment of the body that includes at least the first limb and the second limb for alternating currents at first and second frequencies, wherein the first frequency is lower than the second frequency.

6. An apparatus as claimed in any preceding claim, the apparatus further comprising:
first and second current electrodes that are configured to be attached to the first limb and the second limb of the subject respectively;
a first set of measurement electrodes that are configured to be attached to the first limb; and
a second set of measurement electrodes that are configured to be attached to the second limb.

7. An apparatus as claimed in claim 6, wherein the control unit is configured to:
obtain the measurement of the bioimpedance of the first limb of the subject using the first and second current electrodes and the first set of measurement electrodes;
obtain the measurement of the bioimpedance of the second limb of the subject using the first and second current electrodes and the second set of measurement electrodes; and
obtain the measurement of the bioimpedance of the segment of the body that includes at least the first limb and the second limb using the first and second current electrodes and one of the measurement electrodes in the first set of measurement electrodes and one of the measurement electrodes in the second set of measurement electrodes.

8. An apparatus as claimed in claim 7, further comprising:
a first structure configured to be attached to the first limb, the first structure having embedded or arranged therein the first current electrode and the first set of measurement electrodes;
a second structure configured to be attached to the second limb, the second structure having embedded or arranged therein the second current electrode and the second set of measurement electrodes;
wherein the first and second structures are such that the respective electrodes are in a fixed relationship with each other.

9. A method of operating an apparatus to estimate the fluid content in a subject, the method comprising:
obtaining a measurement of the bioimpedance of a first limb of the subject;
obtaining a measurement of the bioimpedance of a second limb of the subject;
obtaining a measurement of the bioimpedance of the segment of the body that includes at least the first limb and the second limb; and
determining a measure of the fluid content in the first limb using the bioimpedance measurement of the first limb, the bioimpedance measurement of the second limb and the bioimpedance measurement of the segment of the body that includes at least the first limb and the second limb.

10. A method as claimed in claim 9, wherein the step of determining a measure of the fluid content comprises determining a measure of the fluid content in the first limb by normalising the bioimpedance measurements for the first and second limbs using the bioimpedance measurement for the body segment that includes at least the first limb and the second limb.

11. A method as claimed in claim 9, wherein the step of determining a measure of the fluid content comprises:
determining a measure of the fluid content in the first limb by determining the amount of extracellular fluid and/or intracellular fluid in the first and second limbs and the body segment that includes at least the first limb and the second limb from the bioimpedance measurements; and
normalising the measures of extracellular fluid and/or intracellular fluid in the first and second limbs using the measure of extracellular and/or intracellular fluid in the body segment that includes at least the first limb and the second limb.

12. A method as claimed in claim 9, wherein the step of determining a measure of the fluid content comprises:
determining a ratio of extracellular fluid to intracellular fluid in each of the first and second limbs and the body segment that includes at least the first limb and the second limb from the bioimpedance measurements; and
normalising the ratio of extracellular fluid to intracellular fluid for each of the first and second limbs using the ratio of extracellular fluid to intracellular fluid for the body segment that includes at least the first limb and the second limb.

13. A method as claimed in claim 12, wherein the steps of obtaining comprise obtaining measurements of the bioimpedance of the first limb, second limb and the segment of the body that includes at least the first limb and the second limb for alternating currents at first and second frequencies, wherein the first frequency is lower than the second frequency.

14. A method as claimed in claim 13, wherein:
the step of obtaining a measurement of the bioimpedance of the first limb of the subject comprises obtaining the measurement using first and second current electrodes that are attached to a first limb and a second limb of the subject respectively and a first set of measurement electrodes that are attached to the first limb;
the step of obtaining a measurement of the bioimpedance of the second limb of the subject comprises obtaining the measurement using the first and second current electrodes and a second set of measurement electrodes that are attached to the second limb; and
the step of obtaining a measurement of the bioimpedance of the body segment that includes at least the first limb and the second limb comprises obtaining the measurement using the first and second current electrodes, one of the measurement electrodes in the first set of measurement electrodes and one of the measurement electrodes in the second set of measurement electrodes.

15. A computer program product having computer readable code embodied therein, the computer readable code being configured such that, on execution by a suitable computer or processing unit, the computer or processing unit is caused to perform any of the methods as claimed in claim 14.
